(19) United States
(12) Patent Application Publication
(10) Pub. No.: US 2018/0098931 A1
(43) Pub. Date: Apr. 12, 2018

(54) MIXTURE OF SUBSTANCES FOR STYLING AGENT

(71) Applicant: Henkel AG & Co. KGaA, Duesseldorf (DE)

(72) Inventors: Thorsten Knappe, Schenefeld (DE); Marie Meisel, Hemslingen (DE)

(73) Assignee: Henkel AG & Co. KGaA, Duesseldorf (DE)

(21) Appl. No.: 15/666,572

(22) Filed: Aug. 2, 2017

(30) Foreign Application Priority Data
Oct. 12, 2016 (DE) 10 2016 219 838.6

(51) Int. Cl. A61K 8/88; A61K 8/73; A61K 8/04; A61K 8/31; A61Q 5/06

(52) U.S. Cl. CPC A61K 8/88; A61K 8/736; A61K 2800/594; A61K 8/31; A61Q 5/06; A61K 8/046

(57) ABSTRACT

A cosmetic agent for the temporary styling of keratinic fibers, in particular human hair, containing, in a cosmetic support, with respect to its total weight,
a) from about 0.1% to about 2.0% by weight of chitosan or chitosan derivative
b) from about 0.5% to about 7.0% by weight of polyimide, its use and a method employing this agent.

### CROSS-REFERENCE TO RELATED APPLICATION

This application claims priority to German Patent Application No. 10 2016 219 838.6, filed Oct. 12, 2016, which is incorporated herein by reference in its entirety.

### TECHNICAL FIELD

The present disclosure relates to cosmetic agents for the temporary styling of keratinic fibers which contain, in a cosmetic support, a combination of at least one chitosan compound and a polyimide.

The disclosure further relates to methods for the temporary styling of keratinic fibers as well as the use of the cosmetic agent for improving curl retention, and in particular high humidity curl retention.

### BACKGROUND

Agents for temporary shaping are known per se. They usually contain synthetic polymers as the shaping component. Preparations which contain a dissolved or dispersed polymer can be applied to the hair by employing propellant gases or by employing a pump mechanism. Hair gels and hair waxes in particular, however, are not usually applied directly to the hair but are distributed into the hair using a comb or the hands.

The most important property of an agent for the temporary shaping of keratinic fibers, which will also be referred to below as styling agents, is that the freshly modelled treated fibers are provided with as strong a hold as possible. When the keratinic fibers are human hair, this is also known as a strong styling hold or a firm hold of the styling agent.

As a rule, cosmetic styling agents contain individual polymers which are specially tailored to obtain a very specific effect. If different effects are to be obtained, several polymers have to be added. However, if too many different polymers are employed, this can give rise to a whole series of disadvantages. In this regard, problems might arise with the formulation, perhaps because the polymers react with each other or with other components of the agent, leading to precipitation or decomposition. In addition, some specific polymers have a tendency to form stubborn deposits on the skin, and in particular on the hair, which can no longer be completely removed by normal washing, leading to unwanted concentration of the polymer and eventually to strain on the skin or hair.

There is therefore a need for polymers or suitable combinations of fewer polymers which exhibit as many as possible of the desired properties simultaneously.

As an example, in the case of styling agents, the polymers used must provide the treated hair with as strong a hold as possible. In addition to a strong hold, styling agents must satisfy a whole series of other requirements. Broadly, these can be divided into properties on the hair, properties of the respective formulation, for example properties of the mousse, the gel or the sprayed aerosol, and properties which concern the handling of the styling agent; the properties on the hair are particularly important. In this regard, particular mention can be made of moisture resistance, low tack and a well-balanced conditioning effect. Furthermore, a styling agent should be capable of being used for as many hair types as possible and provide the hair with volume and fullness.

### BRIEF SUMMARY

Cosmetic agents are provided. In an exemplary embodiment, a cosmetic agent for the temporary styling of keratinic fibers, in particular human hair, is provided. the cosmetic agent includes, in a cosmetic support, with respect to its total weight,

a) from about 0.1% to about 2.0% by weight of chitosan or chitosan derivative

b) from about 0.5% to about 7.0% by weight of polyimide.

A method for the temporary shaping of keratinic fibers, in particular human hair, is also provided. In an exemplary embodiment, a cosmetic agent is applied to the keratinic fibers. The cosmetic agent includes, in a cosmetic support, with respect to its total weight;

a) from about 0.1% to about 2.0% by weight of chitosan or chitosan derivative

b) from about 0.5% to about 7.0% by weight of polyimide.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Thus, the objective of the present disclosure is to provide agents for the temporary shaping of keratinic fibers, which provide a substantial improvement to the hold of a hairstyle and the feel of the hair (soft feel and elasticity). In particular, the objective of the present disclosure is to provide agents which provide a long-lasting enhancement of the fullness of the hair and the volume of the hair, in particular after washing the hair.

These objectives are achieved by employing a special combination of substances comprising a chitosan and a polyimide. Fixing mousses containing chitosan and polyimide have been disclosed in the US patent application US 2013/0309190 A1, for example.

The agents and methods as contemplated herein and some of their preferred embodiments are defined in the following points:
1. A cosmetic agent for the temporary styling of keratinic fibers, in particular human hair, containing, in a cosmetic support, with respect to its total weight,
a) from about 0.1% to about 2.0% by weight of chitosan or chitosan derivative
b) from about 0.5% to about 7.0% by weight of polyimide.
2. A cosmetic agent according to one of the preceding points, wherein the proportion by weight of the chitosan or chitosan derivative, with respect to the total weight of the cosmetic agent is from about 0.15% to about 1.5% by weight, or from about 0.2% to about 1.0% by weight.
3. A cosmetic agent according to one of the preceding points, wherein the proportion by weight of the polyimide with respect to the total weight of the cosmetic agent is from about 1.0% to about 5.0% by weight, or from about 2.0% to about 4.0% by weight.
4. A cosmetic agent according to one of the preceding points, wherein the polyimide contains at least one structural unit (A1), at least one structural unit (A2), at least one structural unit (A3) and at least one structural unit (A4),

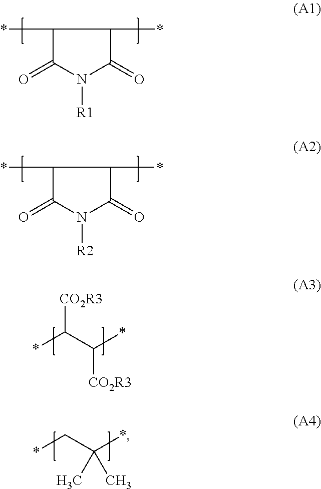

wherein

R1 represents an alkyl residue which may optionally be heterofunctionalized;

R2 represents an alkyl residue which differs from R1 and which may optionally be heterofunctionalized;

R3, independently of each other, represent alkyl residues which may optionally be heterofunctionalized and which differ from R1 and R2.
5. A cosmetic agent according to point 4, wherein R1 in formula (A1) represents a residue —CH(CH₃)CH₂—(OCH(CH₃)CH₂)ₓ(O[CH₂]₂)_{y}OCH₃, in which x and y, independently of each other, have a value of between 1 and 100.
6. A cosmetic agent according to one of points 4 or 5, wherein residue R2 in the structural unit (A2) represents a residue containing an amino group, such as a residue with a tertiary amine.
7. A cosmetic agent according to one of points 4 to 6, wherein at least one of the residues R3 of the structural unit (A3) represents an alkyl residue, such as a (C1 to C4) alkyl residue.
8. A cosmetic agent according to one of the preceding points, wherein the agent contains, with respect to its total weight, from about 0.05% to about 2.0% by weight, or from about 0.1% to about 1.5% by weight, or from about 0.2% to about 1.0% by weight of a C1-C4 carboxylic acid, such as a C1-C4 carboxylic acid selected from the group by formic acid and lactic acid.
9. A cosmetic agent according to one of the preceding points, wherein the agent, with respect to its total weight, further contains from about 0.5% to about 6.0% by weight, or from about 1.0% to about 5.0% by weight, or from about 1.5% to about 4.0% by weight of film-forming polymer.
10. A cosmetic agent according to one of the preceding points, wherein the agent, with respect to its total weight, contains from about 0.1% to about 4.0% by weight, or from about 0.1% to about 3.0% by weight, or from about 0.1% to about 2.0% by weight of vinyl pyrrolidone homopolymer.
11. A cosmetic agent according to one of the preceding points, wherein the agent, with respect to its total weight, contains from about 0.1% to about 4.0% by weight, or from about 0.1% to about 3.0% by weight, or from about 0.1% to about 2.0% by weight of vinyl acetate/vinyl pyrrolidone copolymer.
12. A cosmetic agent according to one of the preceding points, wherein the agent, with respect to its total weight, contains from about 0.1% to about 4.0% by weight, or from about 0.15% to about 2.0% by weight, or from about 0.2% to about 1.0% by weight of N-methylvinylimidazole/vinyl pyrrolidone copolymer.
13. A cosmetic agent according to one of the preceding points, wherein the agent, with respect to its total weight, further contains from about 0.01% to about 2.0% by weight, or from about 0.05% to about 1.0% by weight, or from about 0.1% to about 0.8% by weight of silicone, such as an aminofunctionalized silicone.
14. A cosmetic agent according to one of the preceding points, wherein the agent, with respect to its total weight, further contains from about 0.05% to about 2.0% by weight, or from about 0.1% to about 1.0% by weight, or from about 0.1% to about 0.5% by weight of at least one nonionic surfactant from the group of addition products of from about 30 to about 60 Mol ethylene oxide with castor oil and hydrogenated castor oil, such as at least one nonionic surfactant from the group of compounds with the INCI names steareth-30, ceteareth-30, oleth-30, ceteareth-50, PEG-40 hydrogenated castor oil and PEG-60 hydrogenated castor oil, such as PEG-40 hydrogenated castor oil.

15. A cosmetic agent according to one of the preceding points, wherein the cosmetic agent, with respect to its total weight, further contains from about 0.05% to about 3.0% by weight, or from about 0.1% to about 2.0% by weight, or from about 0.1% to about 1.0% by weight of at least one cationic surfactant.
16. A cosmetic agent according to one of the preceding points, wherein the cosmetic agent contains, with respect to its total weight, from about 60% to about 96% by weight, or from about 70% to about 94% by weight, or from about 80% to about 92% by weight of water or water-alcohol mixture.
17. A cosmetic agent according to one of the preceding points, wherein the cosmetic agent, with respect to its total weight, contains from about 1% to about 15% by weight, or from about 2% to about 12.5% by weight, or from about 3% to about 10% by weight of at least one propellant, such as propane and/or butane.
18. Use of a cosmetic agent according to one of the preceding points, for the temporary styling of keratinic fibers.
19. Use of a cosmetic agent according to one of the preceding points, to improve the volume of the hair, in particular after washing the hair.
20. A method for the temporary shaping of keratinic fibers, in particular human hair, in which a cosmetic agent according to one of the preceding points is applied to the keratinic fibers.

In a first aspect of this application, cosmetic agents are provided for the temporary styling of keratinic fibers, in particular human hair, containing, in a cosmetic support, with respect to its total weight,
a) from about 0.1% to about 2.0% by weight of chitosan or chitosan derivative
b) from about 0.5% to about 7.0% by weight of polyimide.

The substances of the cosmetic agents as contemplated herein are contained in a cosmetic support, preferably in a cosmetic support that contains water, an alcoholic cosmetic support or a hydroalcoholic cosmetic support. For the purposes of temporary hair styling, examples of such supports are lotions, water-in-oil emulsions, oil-in-water emulsions, creams, gels, mousses, pomades, waxes or other preparations which are suitable for application to the hair.

As contemplated herein, it is preferable for the cosmetic support to be an aqueous cosmetic support or a hydroalcoholic cosmetic support. As contemplated herein, the cosmetic support of the agent as contemplated herein more preferably contains water so that the agent contains at least 50% by weight of water—with respect to the total weight of the agent.

The term “hydroalcoholic supports” as used in the context of the present disclosure should be understood to mean aqueous compositions which contain water and also C₁-C₄ alcohol, in particular ethanol or isopropanol, methoxybutanol, benzyl alcohol, ethyldiglycol, 1,2-propyleneglycol or 1,3-propyleneglycol.

A preferred embodiment of the present disclosure is cosmetic agents, with respect to their total weight, that contain from about 60% to about 96% by weight, or from about 70% to about 94% by weight, or from about 80% to about 92% by weight of water or water-alcohol mixture.

A first essential component of the cosmetic agent as contemplated herein is chitosan or chitosan derivative a). Having regard to the manufacturability, applicability and cosmetic action of cosmetic compositions as contemplated herein, it has been shown to be advantageous for the proportion by weight of the chitosan or chitosan derivative, with respect to the total weight of the cosmetic agent to be from about 0.15% to about 1.5% by weight, or from about 0.2% to about 1.0% by weight. The use of a minimum quantity of 0.1% by weight of chitosan has been shown to be essential to the cosmetic action of the agent. Smaller quantities of chitosan do not provide any useful cosmetic effects.

Chitosans are biopolymers and are classified into the hydrocolloid group. Chemically, they are partially deacetylated chitins with different molecular weights. Chitosans are manufactured from chitin, preferably from the shell residues of crustaceans, which are available in large quantities as a cheap starting material. The chitin is usually initially deproteinized by adding bases, demineralized by adding mineral acids and finally deacetylated by adding strong bases, wherein the molecular weights can be distributed over a wide range. Preferably, those types are used which have an average molecular weight (mass average) of from about 800000 to about 1200000 Dalton, a Brookfield viscosity (1% by weight in glycolic acid) of less than 5000 millipascal second (mPas,) a degree of deacetylation in the range of from about 80% to about 88% and an ash content of less than 0.3% by weight.

Typical biopolymers in addition to chitosans in the context of the present disclosure include cationic chitosan derivatives (such as quaternized products) or alkoxylated chitosans as the chitosan derivatives.

Exemplary agents as contemplated herein contain, as the chitosan, at least one neutralization product of chitosan with at least one organic carboxylic acid such as, in particular, formic acid, acetic acid, citric acid, lactic acid, pyrrolidone carboxylic acid, tartaric acid, glycolic acid, nicotinic acid, hydroxyisobutyric acid, hydroxyisovaleric acid or mixtures of these acids. In this regard, as contemplated herein, the organic carboxylic acids are selected from lactic acid, formic acid, pyrrolidone carboxylic acid, nicotinic acid, hydroxyisobutyric acid, hydroxyisovaleric acid or mixtures of these acids. This neutralization product may, for example, be produced in an aqueous medium by adding chitosan and the appropriate organic carboxylic acid.

Examples of suitable chitosans are freely commercially available under the trade names Hydagen® CMF (1% by weight of active substance with 0.4% by weight of glycolic acid, molecular weight from about 500000 to about 5000000 g/mol, Cognis), Hydagen® HCMF (80% deacetylated chitosan, molecular weight from about 50000 to about 1000000 g/mol, Cognis), Kytamer® PC (80% by weight of active substance, chitosan pyrrolidone carboxylate (INCI name: chitosan PCA), Amerchol) and Chitolam® NB/101.

The agents as contemplated herein contain a polyimide as the second essential component.

In an exemplary embodiment, the polyimide contains at least one structural unit (A1), at least one structural unit (A2), at least one structural unit (A3) and at least one structural unit (A4),

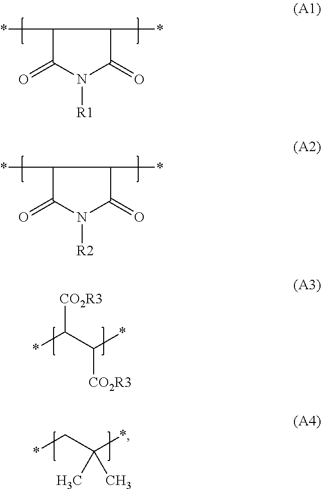

wherein

R1 represents an alkyl residue which may optionally be heterofunctionalized;

R2 represents an alkyl residue which differs from R1 and which may optionally be heterofunctionalized;

R3, independently of each other, represent alkyl residues which may optionally be heterofunctionalized and which differ from R1 and R2.

Having regard to the cosmetic properties of the agents as contemplated herein, it has been shown to be advantageous when:

residue R1 in formula (A1) represents a residue —CH(CH₃)CH₂—(OCH(CH₃)CH₂)ₓ(O[CH₂]₂)_{y}OCH₃, in which x and y, independently of each other, have a value between 1 and 100 and/or
residue R2 in the structural unit (A2) represents a residue containing an amino group, such as a residue with a tertiary amine, and/or
at least one of the residues R3 of the structural unit (A3) represents an alkyl residue, such as a (C1 to C4) alkyl residue.

Exemplary polyimides contain at least 70% by weight, or at least 80% by weight, or least 90% by weight, or at least 95% by weight of structural units (A1), (A2), (A3) and (A4). Exemplary copolymers A are formed entirely of the structural units (A1), (A2), (A3) and (A4).

Some exemplary polyimides are listed below. These polyimides contain at least 70% by weight, or at least 80% by weight, or at least 90% by weight, or at least 95% by weight, or are formed entirely of the structural units (A1), (A2), (A3) and (A4):
A-I) Polyimides with at least one structural unit (A1), at least one structural unit (A2), at least one structural unit (A3) and at least one structural unit (A4),

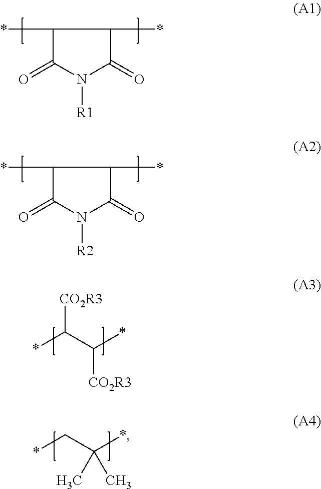

wherein

R1 in formula (A1) represents a residue —CH(CH₃)CH₂—(OCH(CH₃)CH₂)ₓ(O[CH₂]₂)_{y}OCH₃, in which x and y, independently of each other, have a value of between 1 and 100;

R2 in formula (A2) represents a residue —(CH₂)₃—N(CH₃)₂;

R3, independently of each other, represent alkyl residues which may optionally be heterofunctionalized and which differ from R1 and R2.
A-II) Polyimides with at least one structural unit (A1), at least one structural unit (A2), at least one structural unit (A3) and at least one structural unit (A4),

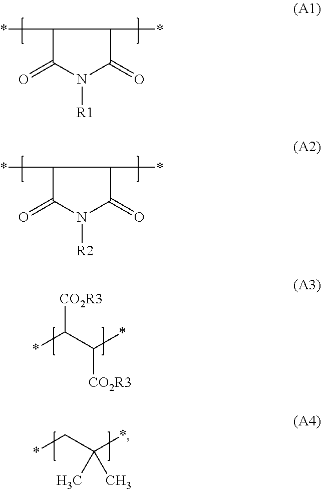

wherein

R1 in formula (A1) represents a residue —CH(CH₃)CH₂—(OCH(CH₃)CH₂)ₓ(O[CH₂]₂)_{y}OCH₃, in which x and y, independently of each other, have a value of between 1 and 100;

R2 represents an alkyl residue which differs from R1 and which may optionally be heterofunctionalized;

one residue R3 in the formula (A3) represents —CH₂CH₃ or —CH₂CH₂CH₃, and one residue R3 in formula (A3) represents H.
A-III) Polyimides with at least one structural unit (A1), at least one structural unit (A2), at least one structural unit (A3) and at least one structural unit (A4),

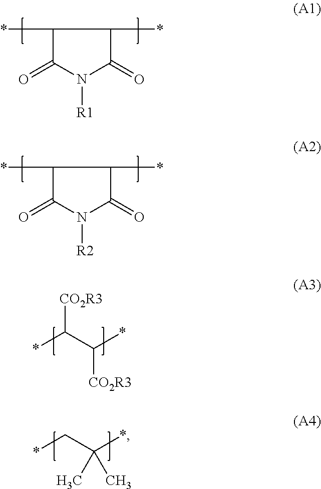

wherein

R1 represents an alkyl residue which may optionally be heterofunctionalized;

R2 in formula (A2) represents a residue —(CH₂)₃—N(CH₃)₂;

one residue R3 in the formula (A3) represents —CH₂CH₃ or —CH₂CH₂CH₃ and one residue R3 in formula (A3) represents H.

Exemplary polyimides are exemplified in that

the residue R1 in formula (A1) represents a residue —CH(CH₃)CH₂—(OCH(CH₃)CH₂)ₓ(O[CH₂]₂)_{y}OCH₃, in which x and y, independently of each other, have a value of between 1 and 100;

the residue R2 in formula (A2) represents a residue —(CH₂)₃—N(CH₃)₂;

one residue R3 represents —CH₂CH₃ and one residue R3 in formula (A3) represents H.

Suitable polyimides are, for example, freely commercially available from ISP under the trade names Aquaflex XL 30 (INCI: polyimide-1).

The proportion by weight of the polyimide with respect to the total weight of the cosmetic agent is advantageously from about 1.0% to about 5.0% by weight, or from about 2.0% to about 4.0% by weight.

In addition to the two components described above, the cosmetic agent as contemplated herein may contain other substances or auxiliary agents, wherein particularly preferred substances or auxiliary agents are those which improve the manufacturability, applicability and/or cosmetic action of these agents.

An exemplary component of the cosmetic agent is constituted by C1-C4 carboxylic acids, the proportion by weight of which with respect to the total weight of the cosmetic agent is from about 0.05% to about 2.0% by weight, or from about 0.1% to about 1.5% by weight, or from about 0.2% to about 1.0% by weight. Exemplary C1-C4 carboxylic acids are selected from the group by formic acid and lactic acid, wherein lactic acid is particularly preferred.

Film-forming polymers constitute a further component of the cosmetic agent. Temporary as well as permanent cationic, anionic, non-ionic or amphoteric polymers are suitable as the film-forming polymers. These film-forming polymers may be of synthetic or natural origins. Exemplary cosmetic agents contain, with respect to their total weight, from about 0.5% to about 6.0% by weight, or from about 1.0% to about 5.0% by weight, or from about 1.5% to about 4.0% by weight of film-forming polymer. This film-forming polymer is different from the chitosan and the polyimide.

Examples of customary film-forming polymers are acrylamide/ammonium acrylate copolymer, acrylamides/DMAPA acrylates/methoxy PEG methacrylate copolymer, acrylamidopropyltrimonium chloride/acrylamide copolymer, acrylamidopropyltrimonium chloride/acrylates copolymer, acrylates/acetoacetoxyethyl methacrylate copolymer, acrylates/acrylamide copolymer, acrylates/ammonium methacrylate copolymer, acrylates/t-butylacrylamide copolymer, acrylates copolymer, acrylates/C1-2 succinates/hydroxyacrylates copolymer, acrylates/lauryl acrylate/stearyl acrylate/ethylamine oxide methacrylate copolymer, acrylates/octylacrylamide copolymer, acrylates/octylacrylamide/diphenyl amodimethicone copolymer, acrylates/stearyl acrylate/ethylamine oxide methacrylate copolymer, acrylates/VA copolymer, acrylates/VP copolymer, adipic acid/diethylenetriamine copolymer, adipic acid/dimethylaminohydroxypropyl diethylenetriamine copolymer, adipic acid/epoxypropyl diethylenetriamine copolymer, adipic acid/isophthalic acid/neopentyl glycol/trimethylolpropane copolymer, allyl stearate/VA copolymer, aminoethylacrylate phosphate/acrylates copolymer, aminoethylpropanediol-acrylates/acrylamide copolymer, aminoethylpropanediol-AMPD-acrylates/diacetone acrylamide copolymer, ammonium VA/acrylates copolymer, AMPD-acrylates/diacetone acrylamide copolymer, AMP-acrylates/allyl methacrylate copolymer, AMP-acrylates/C1-18 alkyl acrylates/C1-8 alkyl acrylamide copolymer, AMP-acrylates/diacetone acrylamide copolymer, AMP-acrylates/dimethylaminoethylmethacrylate copolymer, bacillus/rice bran extract/soybean extract ferment filtrate, bis-butyloxyamodimethicone/PEG-60 copolymer, butyl acrylate/ethylhexyl methacrylate copolymer, butyl acrylate/hydroxypropyl dimethicone acrylate copolymer, butylated PVP, butyl ester of ethylene/MA copolymer, butyl ester of PVM/MA copolymer, calcium/sodium PVM/MA copolymer, corn starch/acrylamide/sodium acrylate copolymer, diethylene glycolamine/epichlorohydrin/piperazine copolymer, dimethicone crosspolymer, diphenyl amodimethicone, ethyl ester of PVM/MA copolymer, hydrolyzed wheat protein/PVP crosspolymer, isobutylene/ethylmaleimide/hydroxyethylmaleimide copolymer, isobutylene/MA copolymer, isobutylmethacrylate/bis-hydroxypropyl dimethicone acrylate copolymer, isopropyl ester of PVM/MA copolymer, lauryl acrylate crosspolymer, lauryl methacrylate/glycol dimethacrylate crosspolymer, MEA-sulfite, methacrylic acid/sodium acrylamidomethyl propane sulfonate copolymer, methacryloyl ethyl betaine/acrylates copolymer, octylacrylamide/acrylates/butylaminoethyl methacrylate copolymer, PEG/PPG-25/25 dimethicone/acrylates copolymer, PEG-8/SMDI copolymer, polyacrylamide, polyacrylate-6, polybeta-alanine/glutaric acid crosspolymer, polybutylene terephthalate, polyester-1, polyethylacrylate, polyethylene terephthalate, polymethacryloyl ethyl betaine, polypentaerythrityl terephthalate, polyperfluoroperhydrophenanthrene, polysilicone-9, polyurethane-1, polyurethane-6, polyurethane-10, polyvinyl acetate, polyvinyl butyral, polyvinylcaprolactam, polyvinyl formamide, polyvinyl imidazolinium acetate, polyvinyl methyl ether, potassium butyl ester of PVM/MA copolymer, potassium ethyl ester of PVM/MA copolymer, PPG-70 polyglyceryl-10 ether, PPG-12/SMDI copolymer, PPG-51/SMDI copolymer, PPG-10 sorbitol, PVM/MA copolymer, PVP, PVP/VA/itaconic acid copolymer, PVP/VA/vinyl propionate copolymer, rhizobian gum, rosin acrylate, shellac, sodium butyl ester of PVM/MA copolymer, sodium ethyl ester of PVM/MA copolymer, sodium polyacrylate, sterculia urens gum, terephthalic acid/isophthalic acid/sodium isophthalic acid sulfonate/glycol copolymer, trimethylolpropane triacrylate, trimethylsiloxysilylcarbamoyl pullulan, VA/crotonates copolymer, VA/crotonates/methacryloxybenzophenone-1 copolymer, VA/crotonates/vinyl neodecanoate copolymer, VA/crotonates/vinyl propionate copolymer, VA/DBM copolymer, VA/vinyl butyl benzoate/crotonates copolymer, vinylamine/vinyl alcohol copolymer, vinyl caprolactam/VP/dimethylaminoethyl methacrylate copolymer, VP/acrylates/lauryl methacrylate copolymer, VP/dimethylaminoethylmethacrylate copolymer, VP/DMAPA acrylates copolymer, VP/hexadecene copolymer, VP/VA copolymer, VP/vinyl caprolactam/DMAPA acrylates copolymer, yeast palmitate and styrene/VP copolymer.

An exemplary group of polymers a3) used in the cosmetic preparation is constituted by vinylpyrrolidone homo- or copolymers B. Examples of exemplary polymers a3) are as follows:

polyvinylpyrrolidones, such as those marketed under the name Luviskol® (BASF),

vinylpyrrolidone/vinylester copolymers, such as those marketed under the trade name Luviskol® (BASF). Luviskol® VA 64 and Luviskol® VA 73, which are each vinylpyrrolidone/vinylacetate copolymers, are exemplary non-ionic polymers.

Because of their cosmetic action in combination with chitosan and polyimide, polyvinylpyrrolidone (INCI name: PVP) as well as vinylpyrrolidone/vinylacetate copolymers (INCI name VP/VA copolymer) are particularly preferred.

In a first embodiment, these cosmetic agents contain, with respect to their total weight, from about 0.1% to about 4.0% by weight, or from about 0.1% to about 3.0% by weight, or from about 0.1% to about 2.0% by weight of vinylpyrrolidone homopolymer.

In a second embodiment, these cosmetic agents contain, with respect to their total weight, from about 0.1% to about 4.0% by weight, or from about 0.1% to about 3.0% by weight, or from about 0.1% to about 2.0% by weight of vinylacetate/vinylpyrrolidone copolymer.

In a third embodiment, these cosmetic agents contain, with respect to their total weight, from about 0.1% to about 4.0% by weight, or from about 0.1% to about 3.0% by weight, or from about 0.1% to about 2.0% by weight of vinylpyrrolidone homopolymer as well as from about 0.1% to about 4.0% by weight, or from about 0.1% to about 3.0% by weight, or from about 0.1% to about 2.0% by weight of vinylacetate/vinylpyrrolidone copolymer.

Cationic polymers form a further group of exemplary film-forming polymers, including cationic polymers with the INCI names polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-39, polyquaternium-45, polyquaternium-46, polyquaternium-47, polyquaternium-48, polyquaternium-49, polyquaternium-50, polyquaternium-55, polyquaternium-56, polyquaternium-68 and polyquaternium-69. Exemplary cosmetic products are exeplified in that the film-forming polymer a3) is selected from the group of cationic polymers with the INCI names polyquaternium-4, polyquaternium-11, polyquaternium-16, polyquaternium-55, polyquaternium-69. Appropriate cosmetic products are exemplified by particularly good cosmetic hair properties, in particular care and styling properties.

Exemplary cationic polymers include

cationic cellulose derivatives, such as those produced by a reaction of hydroxyethylcellulose with a dimethyldiallylammonium reagent (such as dimethyldiallylammonium chloride), optionally in the presence of other reagents. Some exemplary cationic celluloses of these cationic celluloses also include those with the INCI name polyquaternium-4. Appropriate celluloses are marketed, for example, by National Starch with the names Celquat® H 100 and Celquat® L 200;

copolymers of vinyl pyrrolidones with quaternized derivatives of dialkylaminoalkylacrylates and methacrylates, in particular with diethyl sulphate-quaternized copolymers of vinyl pyrrolidone and dimethylaminoethylmethacrylate (INCI name: polyquaternium-11). Appropriate polymers are available, for example, under the trade names Gafquat® 440, Gafquat® 734 and Gafquat® 755 N (ISP);

copolymers of methacryloylaminopropyllauryldimethylammonium chloride with vinyl pyrrolidone and dimethylaminopropylmethacrylamide (INCI name: polyquaternium-55), which are available, for example, from ISP under the trade names Styleze® W-10 or Styleze® W-20;

copolymers of N-vinyl pyrrolidone, N-vinylcaprolactam, N-(3-dimethylaminopropyl)methacrylamide and 3-(methacryloylamino)propyl-lauryl-dimethylammonium chloride (INCI name: polyquaternium-69) which, for example, are marketed by ISP under the trade names Aquastyle® 300 (28-32% by weight of active substance in ethanol-water mixture, molecular weight 350000);

N-methylvinylimidazole/vinyl pyrrolidone copolymers (INCI name: polyquaternium-16) which, for example, are marketed by BASF under the trade names Luviquat® style, Luviquat® FC 370, Luviquat®FC 550, Luviquat® FC 905 and Luviquat® HM 552;

N-methylvinylimidazole/vinyl pyrrolidone copolymers (INCI name: polyquaternium-16) which, for example, are marketed by BASF under the trade names Luviquat® Style, Luviquat® FC 370, Luviquat®FC 550, Luviquat® FC 905 and Luviquat® HM 552.

Exemplary cosmetic agents are those which contain, with respect to their total weight, from about 0.1% to about 4.0% by weight, or from about 0.15% to about 2.0% by weight, or from about 0.2% to about 1.0% by weight of N-methylvinylimidazole/vinyl pyrrolidone copolymer.

Because of their superlative styling properties, exemplary cosmetic agents are those which contain both non-ionic polymers and cationic polymers.

In order to improve the manufacturability, applicability and cosmetic action, the cosmetic agents preferably contain non-ionic surfactants, wherein exemplary cosmetic agents contain, with respect to their total weight, from about 0.05% to about 2.0% by weight, or from about 0.1% to about 1.0% by weight, or from about 0.1% to about 0.5% by weight of at least one nonionic surfactant, such as at least one nonionic surfactant from the group formed by addition products of from about 30 to about 60 Mol ethylene oxide with castor oil and hydrogenated castor oil, advantageously at least one nonionic surfactant from the group of compounds with the INCI names steareth-30, ceteareth-30, oleth-30, ceteareth-50, PEG-40 hydrogenated castor oil and PEG-60 hydrogenated castor oil, in particular PEG-40 hydrogenated castor oil.

Exemplary non-ionic surfactants are PEG derivatives of hydrogenated castor oil which, for example, are obtainable under the name PEG hydrogenated castor oil, for example PEG-30 hydrogenated castor oil, PEG-33 hydrogenated castor oil, PEG-35 hydrogenated castor oil, PEG-36 hydrogenated castor oil, PEG-40 hydrogenated castor oil or PEG-60 hydrogenated castor oil. Exemplary non-ionic surfactants as contemplated herein are those which are selected from the group of PEG derivatives of hydrogenated castor oil, such as from the group of PEG-40 hydrogenated castor oil and PEG-60 hydrogenated castor oil, in particular PEG-40 hydrogenated castor oil.

Cationic surfactants form a further exemplary component of the cosmetic agents as contemplated herein. Exemplary cationic surfactants are selected from quaternary ammonium compounds, esterquats and amidoamines. The cationic surfactants are contained in the cosmetic agent, with respect to its total weight, in quantities of from about 0.05% to about 3.0% by weight, or from about 0.1% to about 2.0% by weight, or from about 0.1% to about 1.0% by weight. Cationic surfactants from the quaternary ammonium compound group are particularly preferred.

Exemplary quaternary ammonium compounds are ammonium halides, in particular chlorides and bromides, such as alkyltrimethylammonium chloride, dialkyldimethylammonium chloride and trialkylmethylammonium chloride, for example cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as imidazolium compounds known by their INCI names quaternium-27 and quaternium-83. The long alkyl chains of the surfactants mentioned above preferably contain 10 to 18 carbon atoms. More exemplary cosmetic agents as contemplated herein contain, with respect to their total weight, from about 0.05% to about 3.0% by weight, or from about 0.1% to about 2.0% by weight, or from about 0.1% to about 1.0% by weight of (C12 to C18)-alkyltrimethylammonium salt(s).

Additional care products may in particular be cited as further suitable active substances or auxiliary agents.

A first group of exmplary care components is formed by silicones, in particular

alkoxylated dimethicones, preferably from the group formed by ethoxylated/propoxylated dimethicones;

aminofunctionalized silicones.

The following examples belong to the alkoxylated dimethicones group:

ethoxylated dimethicones with the INCI name PEG-x dimethicones with x=2 to 20, or 3 to 17, or 11 or 12;

ethoxylated dimethicones with the INCI name bis-PEG-y dimethicones with x=3 to 25, or 4 to 20;

ethoxylated/propoxylated dimethicones with the INCI name PEG/PPG a/b dimethicones, wherein a and b independently of each other represent numbers from 2 to 30, or 12 to 24, or 14 to 20;

ethoxylated/propoxylated dimethicones with the INCI name bis-PEG/PPG-c/d dimethicones, wherein c and d independently of each other represent numbers from 10 to 25, or 14 to 20, or 14 to 16;

ethoxylated/propoxylated dimethicones with the INCI name bis-PEG/PPG-e/f PEG/PPG g/h dimethicones, wherein e, f, g and h, independently of each other, represent numbers from 10 to 20, or from 14 to 18, or 16.

The group formed by aminofunctionalized silicones which have at least one, optionally substituted, amino group, are known under the INCI system as amodimethicones.

Preferred cosmetic preparations contain one or more aminofunctionalized silicones with formula M(RₐQ_{b}SiO_{(4-a-b)/2})ₓ(R_{c}SiO_{(4-c)/2})_{y}M (Si-1), wherein in the above formula:
R is a hydrocarbon or a hydrocarbon residue containing 1 to approximately 6 carbon atoms, such as methyl,
Q is a polar residue with general formula —R¹HZ, such as a residue with formula —CH₂CH₂CH₂NHCH₂CH₂NH₂,

wherein
R¹ is a divalent binding group which is bonded to hydrogen and the residue Z, composed of carbon atoms and hydrogen atoms, carbon, hydrogen and oxygen atoms or carbon, hydrogen and nitrogen atoms, and
Z is an organic aminofunctionalized residue which contains at least one aminofunctional group, and it is optionally a —NHCH₂CH₂NH₂ residue or a residue with formula —CH₂CH₂CH₂NHCH₂CH₂NH₂;
a takes values in the range from approximately 0 to approximately 2,
b takes values in the range from approximately 1 to approximately 3,
a+b is less than or equal to 3, and
c is a number in the range from approximately 1 to approximately 3, and
x is a number in the range from 1 to approximately 2000, or from approximately 3 to approximately 50, or from approximately 3 to approximately 25, and
y is a number in the range from approximately 20 to approximately 10000, or from approximately 125 to approximately 10000, or from approximately 150 to approximately 1000, and
M is a suitable silicone end group, as is known in the art, preferably trimethylsiloxy.

Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂ CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄— and —(CH₂)₃C(O)SCH₂CH₂—.

Exemplary cosmetic preparations as contemplated herein contain an aminofunctionalized silicone with formula R′ₐG₃₋ₐ-Si(OSiG₂)ₙ—(OSiG_{b}R′_{2-b})ₘ—O—SiG₃₋ₐ-R′ₐ (Si-2), wherein:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a represents a number between 0 and 3, in particular 0;
b represents a number between 0 and 1, in particular 1,
m and n are numbers the sum of which (m+n) is between 1 and about 2000, preferably between about 50 and about 150, wherein n preferably takes values from 0 to about 1999 or from about 49 to about 149 and m preferably takes values from 1 to about 2000, in particular from 1 to about 10,
R′ is a monovalent residue selected from

-Q-N(R″)—CH₂—CH₂—N(R″)₂

-Q-N(R″)₂

-Q-N⁻(R″)₃A⁻

-Q-N⁻H(R″)₂ A⁻

-Q-N⁺H₂(R″)A⁻

-Q-N(R″)—CH₂—CH₂—N⁺R″H₂A⁻,

wherein each Q represents a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
R″ represents identical or different residues from the group formed by —H, -phenyl, -benzyl, —CH₂—CH(CH₃)Ph, C₁₋₂₀-alkyl residues, such as —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃, and A represents an anion which is optionally selected from chloride, bromide, iodide or methosulphate.

Exemplary cosmetic agents contain at least one aminofunctional silicone with formula (Si1-a)

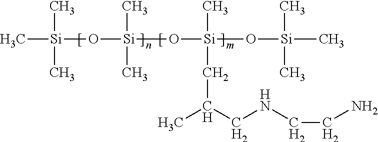

wherein m and n are numbers the sum of which (m+n) is between 1 and about 2000, or between about 50 and about 150, wherein n preferably takes values from 0 to about 1999 and in particular from about 49 to about 149 and m preferably takes values from 1 to about 2000, in particular from 1 to 10.

In a further exemplary embodiment, cosmetic preparations contain an aminofunctional silicone with formula (Si-1b)

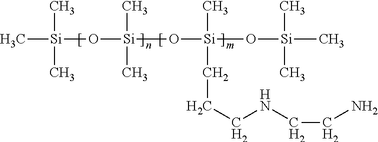

wherein m and n are numbers the sum of which (m+n) is between 1 and about 2000, or between about 50 and about 150, wherein n preferably takes values from 0 to about 1999, or from about 49 to about 149 and m preferably takes values from 1 to about 2000, or from 1 to 10.

These silicones with formula (Si1-a) and (Si1-b) are denoted amodimethicones according to the INCI system. Corresponding amodimethicones are, for example, available in the form of an emulsion under the trade name Dow Corning 939 or under the trade name Dow Corning 949.

In an alternative embodiment, the aminofunctionalized silicone is a functionalized amodimethicone. Appropriate functionalized amodimethicones are described, for example, by the formula (Si-1c)

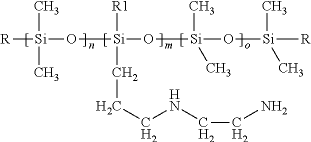

wherein
R represents —OH, an (optionally ethoxylated and/or propoxylated) (C₁ to C₂₀) alkoxy group or a —CH₃ group,
R1 represents —OH, a (C₁ to C₂₀) alkoxy group or a —CH₃ group and
m, n1 and n2 are numbers, the sum of which (m+n1+n2) is between 1 and about 2000, or between 50 and 150, wherein the sum (n1+n2) optionally takes values from 0 to about 1999 and in particular from about 49 to about 149 and m optionally takes values from 1 to about 2000, in particular from 1 to 10.

Exemplary examples of aminofunctional silicones are from the group formed by hydroxylamino-modified silicones, in which in the above formula (Si-1b), the residue R represents —OH. Appropriate silicones with the INCI name amodimethicone are available, for example, under the trade name Wacker Belsil ADM 652.

If the residue R represents an (optionally ethoxylated and/or propoxylated) (C₁ to C₂₀) alkoxy group, then, for example, this general formula (Si-1b) includes bis-(C13-15 alkoxy) PG amodimethicones (example of trade name: DC 8500 from Dow Corning), trideceth-9 PG-amodimethicones (example of trade name: Silcare Silicone SEA from Clariant).

Irrespectively of which aminofunctionalized silicone is used, exemplary cosmetic agents as contemplated herein are those which contain an aminofunctional silicone with an amine number of more than 0.25 milliequivalent per gram (meq/g,) or more than 0.3 meq/g, or more than 0.4 meq/g. The term “amine number” as used here means milli-equivalents of amine per gram of aminofunctional silicone. It can be determined by titration and also given in the units of mg KOH/g.

Exemplary cosmetic agents are exemplified in that these agents, with respect to their total weight, further contain from about 0.01% to about 2.0% by weight, or from about 0.05% to about 1.0% by weight, or from about 0.1% to about 0.8% by weight of silicone, such as an aminofunctional silicone.

The agent may, for example, contain at least one protein hydrolysate and/or a derivative thereof as the care product. Protein hydrolysates are product mixtures which are obtained by the acidic, basic or enzymatically catalysed decomposition of proteins. The term “protein hydrolysate” as used in the present disclsoure should also be understood to include total hydrolysates as well as individual amino acids and their derivatives, as well as mixtures of the various amino acids. The molecular weight of the protein hydrolysates used as contemplated herein is between about 75, the molecular weight of glycine, and about 200000; or, the molecular weight is from about 75 to about 50000, or from about 75 to about 20000 Dalton.

The agent as contemplated herein may also contain at least one vitamin, a provitamin, a vitamin precursor and/or one of its derivatives as the care product. In this regard, as contemplated herein, those vitamins, provitamins and vitamin precursors are preferred which are usually categorized into the groups A, B, C, E, F and H.

Other care products are panthenol, caffeine, nicotinamide and sorbitol.

The agent as contemplated herein may also contain at least one plant extract as the care product, as well as mono- or oligosaccharides and/or lipids.

The composition of some exemplary cosmetic agents can be obtained from the following tables (information as the % by weight with respect to the total weight of the cosmetic agent, unless indicated otherwise).

| | Formula 1 | Formula 2 | Formula 3 | Formula 4 |
|---|---|---|---|---|
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Water | 60 to 96 | 70 to 94 | 70 to 94 | 80 to 92 |
| Other | qs 100 | qs 100 | qs 100 | qs 100 |
| ingredients | | | | |
| | Formula 6 | Formula 7 | Formula 8 | Formula 9 |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Water | 60 to 96 | 70 to 94 | 70 to 94 | 80 to 92 |
| Other | qs 100 | qs 100 | qs 100 | qs 100 |
| ingredients | | | | |
| | Formula 11 | Formula 12 | Formula 13 | Formula 14 |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| C₁-C₄ | 0.05 to 2.0 | 0.05 to 2.0 | 0.1 to 1.5 | 0.2 to 1.0 |
| carboxylic | | | | |
| acid | | | | |
| Water | 60 to 96 | 70 to 94 | 70 to 94 | 80 to 92 |
| Other | qs 100 | qs 100 | qs 100 | qs 100 |
| ingredients | | | | |
| | Formula 16 | Formula 17 | Formula 18 | Formula 19 |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| C₁-C₄ | 0.05 to 2.0 | 0.05 to 2.0 | 0.1 to 1.5 | 0.2 to 1.0 |
| carboxylic | | | | |
| acid | | | | |
| Water | 60 to 96 | 70 to 94 | 70 to 94 | 80 to 92 |
| Other | qs 100 | qs 100 | qs 100 | qs 100 |
| ingredients | | | | |
| | Formula 21 | Formula 22 | Formula 23 | Formula 24 |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Lactic acid | 0.05 to 2.0 | 0.05 to 2.0 | 0.1 to 1.5 | 0.2 to 1.0 |
| Water | 60 to 96 | 70 to 94 | 70 to 94 | 80 to 92 |
| Other | qs 100 | qs 100 | qs 100 | qs 100 |
| ingredients | | | | |
| | Formula 26 | Formula 27 | Formula 28 | Formula 29 |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Lactic acid | 0.05 to 2.0 | 0.05 to 2.0 | 0.1 to 1.5 | 0.2 to 1.0 |
| Water | 60 to 96 | 70 to 94 | 70 to 94 | 80 to 92 |
| Other | qs 100 | qs 100 | qs 100 | qs 100 |
| ingredients | | | | |
| | Formula 31 | Formula 32 | Formula 33 | Formula 34 |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Film-forming | 0.5 to 6.0 | 0.5 to 6.0 | 1.0 to 5.0 | 1.5 to 4.0 |
| polymer | | | | |
| Water | 60 to 96 | 70 to 94 | 70 to 94 | 80 to 92 |
| Other | qs 100 | qs 100 | qs 100 | qs 100 |
| ingredients | | | | |
| | Formula 36 | Formula 37 | Formula 38 | Formula 39 |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Film-forming | 0.5 to 6.0 | 0.5 to 6.0 | 1.0 to 5.0 | 1.5 to 4.0 |
| polymer | | | | |
| Water | 60 to 96 | 70 to 94 | 70 to 94 | 80 to 92 |
| Other | qs 100 | qs 100 | qs 100 | qs 100 |
| ingredients | | | | |
| | Formula 41 | Formula 42 | Formula 43 | Formula 44 |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Polyvinyl- | 0.1 to 4.0 | 0.1 to 4.0 | 0.1 to 3.0 | 0.1 to 2.0 |
| pyrrolidone | | | | |
| Water | 60 to 96 | 70 to 94 | 70 to 94 | 80 to 92 |
| Other | qs 100 | qs 100 | qs 100 | qs 100 |
| ingredients | | | | |
| | Formula 46 | Formula 47 | Formula 48 | Formula 49 |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Polyvinyl- | 0.1 to 4.0 | 0.1 to 4.0 | 0.1 to 3.0 | 0.1 to 2.0 |
| pyrrolidone | | | | |
| Water | 60 to 96 | 70 to 94 | 70 to 94 | 80 to 92 |
| Other | qs 100 | qs 100 | qs 100 | qs 100 |
| ingredients | | | | |
| | Formula 51 | Formula 52 | Formula 53 | Formula 54 |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Vinyl | 0.1 to 4.0 | 0.1 to 4.0 | 0.1 to 3.0 | 0.1 to 2.0 |
| acetate/ | | | | |
| vinyl | | | | |
| pyrrolidone | | | | |
| copolymer | | | | |
| Water | 60 to 96 | 70 to 94 | 70 to 94 | 80 to 92 |
| Other | qs 100 | qs 100 | qs 100 | qs 100 |
| ingredients | | | | |
| | Formula 56 | Formula 57 | Formula 58 | Formula 59 |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Vinyl | 0.1 to 4.0 | 0.1 to 4.0 | 0.1 to 3.0 | 0.1 to 2.0 |
| acetate/ | | | | |
| vinyl | | | | |
| pyrrolidone | | | | |
| copolymer | | | | |
| Water | 60 to 96 | 70 to 94 | 70 to 94 | 80 to 92 |
| Other | qs 100 | qs 100 | qs 100 | qs 100 |
| ingredients | | | | |
| | Formula 61 | Formula 62 | Formula 63 | Formula 64 |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| N- | 0.1 to 4.0 | 0.1 to 4.0 | 0.15 to 2.0 | 0.2 to 1.0 |
| methylvinyl- | | | | |
| imidazole/ | | | | |
| vinyl | | | | |
| pyrrolidone | | | | |
| copolymer | | | | |
| Water | 60 to 96 | 70 to 94 | 70 to 94 | 80 to 92 |
| Other | qs 100 | qs 100 | qs 100 | qs 100 |
| ingredients | | | | |
| | Formula 66 | Formula 67 | Formula 68 | Formula 69 |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| N- | 0.1 to 4.0 | 0.1 to 4.0 | 0.15 to 2.0 | 0.2 to 1.0 |
| methylvinyl- | | | | |
| imidazole/ | | | | |
| vinyl | | | | |
| pyrrolidone | | | | |
| copolymer | | | | |
| Water | 60 to 96 | 70 to 94 | 70 to 94 | 80 to 92 |
| Other | qs 100 | qs 100 | qs 100 | qs 100 |
| ingredients | | | | |
| | Formula 71 | Formula 72 | Formula 73 | Formula 74 |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Other | qs 100 | qs 100 | qs 100 | qs 100 |
| ingredients | | | | |
| | Formula 76 | Formula 77 | Formula 78 | Formula 79 |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| PEG-40 | 0.05 to 2.0 | 0.05 to 2.0 | 0.1 to 1.0 | 0.1 to 0.5 |
| hydrogenated | | | | |
| castor oil | | | | |
| Water | 60 to 96 | 70 to 94 | 70 to 94 | 80 to 92 |
| Other | qs 100 | qs 100 | qs 100 | qs 100 |
| ingredients | | | | |
| | Formula 81 | Formula 82 | Formula 83 | Formula 84 |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Film-forming | 0.5 to 6.0 | 0.5 to 6.0 | 1.0 to 5.0 | 1.5 to 4.0 |
| polymer | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Other | qs 100 | qs 100 | qs 100 | qs 100 |
| ingredients | | | | |
| | Formula 86 | Formula 87 | Formula 88 | Formula 89 |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Polyvinyl- | 0.1 to 4.0 | 0.1 to 4.0 | 0.1 to 3.0 | 0.1 to 2.0 |
| pyrrolidone | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Other | qs 100 | qs 100 | qs 100 | qs 100 |
| ingredients | | | | |
| | Formula 91 | Formula 92 | Formula 93 | Formula 94 |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Vinyl | 0.1 to 4.0 | 0.1 to 4.0 | 0.1 to 3.0 | 0.1 to 2.0 |
| acetate/ | | | | |
| vinyl | | | | |
| pyrrolidone | | | | |
| copolymer | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Other | qs 100 | qs 100 | qs 100 | qs 100 |
| ingredients | | | | |
| | Formula 95 | Formula 97 | Formula 98 | Formula 99 |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| N- | 0.1 to 4.0 | 0.1 to 4.0 | 0.15 to 2.0 | 0.2 to 1.0 |
| methylvinyl- | | | | |
| imidazole/ | | | | |
| vinyl | | | | |
| pyrrolidone | | | | |
| copolymer | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Other | qs 100 | qs 100 | qs 100 | qs 100 |
| ingredients | | | | |
| | Formula 101 | Formula 102 | Formula 103 | Formula 104 |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Polyvinyl- | 0.1 to 4.0 | 0.1 to 4.0 | 0.1 to 3.0 | 0.1 to 2.0 |
| pyrrolidone | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Other | qs 100 | qs 100 | qs 100 | qs 100 |
| ingredients | | | | |
| | Formula 106 | Formula 107 | Formula 108 | Formula 109 |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Vinyl | 0.1 to 4.0 | 0.1 to 4.0 | 0.1 to 3.0 | 0.1 to 2.0 |
| acetate/ | | | | |
| vinyl | | | | |
| pyrrolidone | | | | |
| copolymer | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Other | qs 100 | qs 100 | qs 100 | qs 100 |
| ingredients | | | | |
| | Formula 111 | Formula 112 | Formula 113 | Formula 114 |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| N- | 0.1 to 4.0 | 0.1 to 4.0 | 0.15 to 2.0 | 0.2 to 1.0 |
| methylvinyl- | | | | |
| imidazole/ | | | | |
| vinyl | | | | |
| pyrrolidone | | | | |
| copolymer | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Other | qs 100 | qs 100 | qs 100 | qs 100 |
| ingredients | | | | |

In accordance with a further exemplary embodiment, the cosmetic agents as contemplated herein are in the form of a mousse. In this regard, the agents as contemplated herein are packaged in a dispensing device which constitutes either a compressed gas container additionally filled with a propellant (aerosol container), or a non-aerosol container. The compressed gas container, with the aid of which a product is distributed by employing the internal gas pressure of the container via a valve, is defined as an “aerosol container”. In contrast to this definition, a “non-aerosol container” is a container which is under normal pressure, with the aid of which a product is distributed by mechanical action using a pump or squeezing system. In this embodiment the cosmetic agent as contemplated herein is in the form of an aerosol mousse in an aerosol container. The agent as contemplated herein therefore optionally additionally contains at least one propellant. Agents as contemplated herein which are in the form of an aerosol product can be manufactured in the normal manner. As a rule, all of the components of the agent as contemplated herein with the exception of the propellant are placed in a suitable pressure-tight container. This is then sealed with a valve. Finally, the desired quantity of propellant is added using the usual techniques.

In the embodiment as an aerosol mousse, the propellants suitable for use in the present disclosure are, for example, selected from N₂O, dimethylether, CO₂, air, alkanes containing from 3 to 5 carbon atoms such as propane, n-butane, iso-butane, n-pentane and iso-pentane, and mixtures thereof.

In accordance with the embodiment in the form of an aerosol mousse, the cited alkanes, mixtures of the cited alkanes or mixtures of the cited alkanes with dimethylether are used as the only propellant. However, the present disclosure also expressly encompasses the simultaneous use of propellants of the chlorofluorocarbon nature, in particular fluorocarbons. Dimethylether, propane, n-butane, iso-butane and mixtures thereof are preferred. Exemplary mixtures of propane and butane are used as the only propellant in proportions by weight of propane to butane of from about 70:30 to about 15:85. As contemplated herein, the term “butane” should be understood to mean n-butane, iso-butane and mixtures of n-butane and iso-butane.

The propellants in the cosmetic agents as contemplated herein are optionally used in a quantity of from about 1% to about 15% by weight, or from about 2% to about 12.5% by weight, or from about 3% to about 10% by weight—with respect to the total weight of the agent—wherein propane, butane and propane/butane mixtures are preferred.

The compositions of further exemplary cosmetic compositions can be obtained from the following tables (information as the % by weight with respect to the total weight of the composition, unless indicated otherwise).

| | Formula 1a | Formula 2a | Formula 3 a | Formula 4a |
|---|---|---|---|---|
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 6a | Formula 7a | Formula 8a | Formula 9a |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 11a | Formula 12a | Formula 13a | Formula 14a |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| C₁-C₄ | 0.05 to 2.0 | 0.05 to 2.0 | 0.1 to 1.5 | 0.2 to 1.0 |
| carboxylic | | | | |
| acid | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 16a | Formula 17a | Formula 18a | Formula 19a |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| C₁-C₄ | 0.05 to 2.0 | 0.05 to 2.0 | 0.1 to 1.5 | 0.2 to 1.0 |
| carboxylic | | | | |
| acid | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 21a | Formula 22a | Formula 23a | Formula 24a |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Lactic acid | 0.05 to 2.0 | 0.05 to 2.0 | 0.1 to 1.5 | 0.2 to 1.0 |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 26a | Formula 27a | Formula 28a | Formula 29a |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Lactic acid | 0.05 to 2.0 | 0.05 to 2.0 | 0.1 to 1.5 | 0.2 to 1.0 |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 31a | Formula 32a | Formula 33a | Formula 34a |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Film-forming | 0.5 to 6.0 | 0.5 to 6.0 | 1.0 to 5.0 | 1.5 to 4.0 |
| polymer | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 36a | Formula 37a | Formula 38a | Formula 39a |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Film-forming | 0.5 to 6.0 | 0.5 to 6.0 | 1.0 to 5.0 | 1.5 to 4.0 |
| polymer | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 41a | Formula 42a | Formula 43a | Formula 44a |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Polyvinyl- | 0.1 to 4.0 | 0.1 to 4.0 | 0.1 to 3.0 | 0.1 to 2.0 |
| pyrrolidone | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 46a | Formula 47a | Formula 48a | Formula 49a |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Polyvinyl- | 0.1 to 4.0 | 0.1 to 4.0 | 0.1 to 3.0 | 0.1 to 2.0 |
| pyrrolidone | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 51a | Formula 52a | Formula 53a | Formula 54a |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Vinyl | 0.1 to 4.0 | 0.1 to 4.0 | 0.1 to 3.0 | 0.1 to 2.0 |
| acetate/ | | | | |
| vinyl | | | | |
| pyrrolidone | | | | |
| copolymer | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 56a | Formula 57a | Formula 58a | Formula 59a |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Vinyl | 0.1 to 4.0 | 0.1 to 4.0 | 0.1 to 3.0 | 0.1 to 2.0 |
| acetate/ | | | | |
| vinyl | | | | |
| pyrrolidone | | | | |
| copolymer | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 61a | Formula 62a | Formula 63a | Formula 64a |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| N- | 0.1 to 4.0 | 0.1 to 4.0 | 0.15 to 2.0 | 0.2 to 1.0 |
| methylvinyl- | | | | |
| imidazole/ | | | | |
| vinyl | | | | |
| pyrrolidone | | | | |
| copolymer | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 66a | Formula 67a | Formula 68a | Formula 69a |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| N- | 0.1 to 4.0 | 0.1 to 4.0 | 0.15 to 2.0 | 0.2 to 1.0 |
| methylvinyl- | | | | |
| imidazole/ | | | | |
| vinyl | | | | |
| pyrrolidone | | | | |
| copolymer | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 71a | Formula 72a | Formula 73a | Formula 74a |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 76a | Formula 77a | Formula 78a | Formula 79a |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| PEG-40 | 0.05 to 2.0 | 0.05 to 2.0 | 0.1 to 1.0 | 0.1 to 0.5 |
| hydrogenated | | | | |
| castor oil | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 81a | Formula 82a | Formula 83a | Formula 84a |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Film-forming | 0.5 to 6.0 | 0.5 to 6.0 | 1.0 to 5.0 | 1.5 to 4.0 |
| polymer | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 86a | Formula 87a | Formula 88a | Formula 89a |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Polyvinyl- | 0.1 to 4.0 | 0.1 to 4.0 | 0.1 to 3.0 | 0.1 to 2.0 |
| pyrrolidone | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 91a | Formula 92a | Formula 93a | Formula 94a |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| Vinyl | 0.1 to 4.0 | 0.1 to 4.0 | 0.1 to 3.0 | 0.1 to 2.0 |
| acetate/ | | | | |
| vinyl | | | | |
| pyrrolidone | | | | |
| copolymer | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula 95a | Formula 97a | Formula 98a | Formula 99a |
| Chitosan | 0.1 to 2.0 | 0.1 to 2.0 | 0.15 to 1.5 | 0.2 to 1.0 |
| Polyimide | 0.5 to 7.0 | 1.0 to 5.0 | 1.0 to 5.0 | 2.0 to 4.0 |
| N- | 0.1 to 4.0 | 0.1 to 4.0 | 0.15 to 2.0 | 0.2 to 1.0 |
| methylvinyl- | | | | |
| imidazole/ | | | | |
| vinyl | | | | |
| pyrrolidone | | | | |
| copolymer | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula | Formula | Formula | Formula |
| | 101a | 102a | 103a | 104a |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Polyvinyl- | 0.1 to 4.0 | 0.1 to 4.0 | 0.1 to 3.0 | 0.1 to 2.0 |
| pyrrolidone | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula | Formula | Formula | Formula |
| | 106a | 107a | 108a | 109a |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| Vinyl | 0.1 to 4.0 | 0.1 to 4.0 | 0.1 to 3.0 | 0.1 to 2.0 |
| acetate/ | | | | |
| vinyl | | | | |
| pyrrolidone | | | | |
| copolymer | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |
| | Formula | Formula | Formula | Formula |
| | 111a | 112a | 113a | 114a |
| Chitosan | 0.1 to 2.0 | 0.1 to 2.0 | 0.15 to 1.5 | 0.2 to 1.0 |
| Polyimide-1 | 0.5 to 7.0 | 1.0 to 5.0 | 1.0 to 5.0 | 2.0 to 4.0 |
| N- | 0.1 to 4.0 | 0.1 to 4.0 | 0.15 to 2.0 | 0.2 to 1.0 |
| methylvinyl- | | | | |
| imidazole/ | | | | |
| vinyl | | | | |
| pyrrolidone | | | | |
| copolymer | | | | |
| Non-ionic | 0.5 to 6.0 | 0.5 to 6.0 | 1.0 to 5.0 | 1.5 to 4.0 |
| surfactant | | | | |
| Water | 60 to 96 | 70 to 94 | 70 to 94 | 80 to 92 |
| Propellant, | 1.0 to 15 | 2.0 to 12.5 | 2.0 to 12.5 | 3.0 to 10 |
| in | | | | |
| particular | | | | |
| propane/ | | | | |
| butane | | | | |
| Other | qs 100 | qs 100 | qs 100 | qs 100 |
| ingredients | | | | |

As already mentioned, the cosmetic compositions as contemplated herein are of particular application for the temporary styling of keratinic fibers and to improve the volume of the hair, in particular after washing the hair. The corresponding uses of the agents as contemplated herein therefore constitute further objectives of this application.

A final objective of the application is constituted by a method for the temporary shaping of keratinic fibers, in particular human hair, in which a cosmetic agent as contemplated herein is applied to the keratinic fibers.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent for the temporary styling of keratinic fibres, in particular human hair, comprising, in a cosmetic support, with respect to its total weight,
a) from about 0.1% to about 2.0% by weight of chitosan or chitosan derivative; and
b) from about 0.5% to about 7.0% by weight of polyimide.

2. The cosmetic agent as claimed in claim 1, wherein the proportion by weight of the chitosan or chitosan derivative with respect to the total weight of the cosmetic agent is from about 0.15% to about 1.5% by weight.

3. A cosmetic agent as claimed in claim 1, wherein the proportion by weight of the polyimide with respect to the total weight of the cosmetic agent is from about 1.0% to about 5.0% by weight.

4. A cosmetic agent as claimed in claim 1, wherein the polyimide comprises at least one structural unit (A1), at least one structural unit (A2), at least one structural unit (A3) and at least one structural unit (A4), 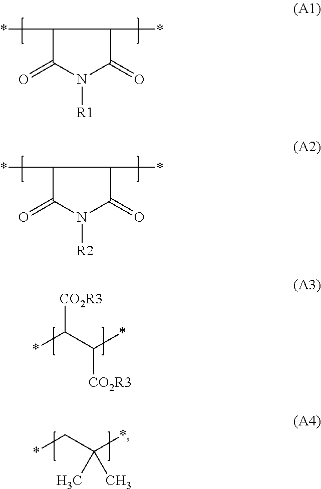
wherein
R1 represents an alkyl residue which may optionally be heterofunctionalized;
R2 represents an alkyl residue which differs from R1 and which may optionally be heterofunctionalized; and
R3, independently of each other, represent alkyl residues which may optionally be heterofunctionalized and which differ from R1 and R2.

5. A cosmetic agent as claimed in claim 1, wherein the agent, with respect to its total weight, further comprises from about 0.5% to about 6.0% by weight of film-forming polymer.

6. A cosmetic agent as claimed in claim 1, wherein the cosmetic agent, with respect to its total weight, comprises from about 60% to about 96% by weight of water or water-alcohol mixture.

7. A cosmetic agent as claimed in claim 1, wherein the cosmetic agent, with respect to its total weight, comprises from about 1% to about 15% by weight at least one propellant.

8. A cosmetic agent as claimed in claim 1, wherein the cosmetic agent is utilized for the temporary styling of keratinic fibres.

9. A cosmetic agent as claimed in claim 1, wherein the cosmetic agent is utilized to improve the volume of the hair, in particular after washing the hair.

10. A method for the temporary shaping of keratinic fibres, in particular human hair, in which a cosmetic agent is applied to the keratinic fibers, wherein the cosmetic agent comprises, in a cosmetic support, with respect to its total weight:
a. from about 0.1% to about 2.0% by weight of chitosan or chitosan derivative; and
b. from about 0.5% to about 7.0% by weight of polyimide.

11. The cosmetic agent as claimed in claim 1, wherein the proportion by weight of the chitosan or chitosan derivative with respect to the total weight of the cosmetic agent is from about 0.2% to about 1.0% by weight.

12. The cosmetic agent as claimed in claim 11 wherein the chitosan or chitosan derivative is chitosan.

13. A cosmetic agent as claimed in claim 1, wherein the proportion by weight of the polyimide with respect to the total weight of the cosmetic agent is from about 2.0% to about 4.0% by weight.

14. A cosmetic agent as claimed in claim 1, wherein the agent, with respect to its total weight, further comprises from about 1.0% to about 5.0% by weight of film-forming polymer.

15. A cosmetic agent as claimed in claim 1, wherein the agent, with respect to its total weight, further comprises from about 1.5% to about 4.0% by weight of film-forming polymer.

16. A cosmetic agent as claimed in claim 7 wherein the at least one propellant comprises propane and/or butane.

17. A cosmetic agent as claimed in claim 1, wherein the cosmetic agent, with respect to its total weight, comprises from about 70% to about 94% by weight of water or water-alcohol mixture.

18. A cosmetic agent as claimed in claim 1, wherein the cosmetic agent, with respect to its total weight, comprises from about from about 80% to about 92% by weight of water or water-alcohol mixture.

19. A cosmetic agent as claimed in claim 1, wherein the cosmetic agent, with respect to its total weight, comprises from about 2% to about 12.5% by weight and of at least one propellant.

20. A cosmetic agent as claimed in claim 1, wherein the cosmetic agent, with respect to its total weight, comprises from about 3% to about 10% by weight of at least one propellant, wherein the at least one propellant comprises propane and/or butane.
